(19) United States
(12) Patent Application Publication
(10) Pub. No.: US 2010/0267112 A1
(43) Pub. Date: Oct. 21, 2010

(54) USE OF PKS 13 PROTEIN CODING FOR CONDENSASE OF MYCOLIC ACIDS OF MYCOBACTERIA AND RELATED STRAINS AS AN ANTIBIOTICS TARGET

(75) Inventors: CHRISTOPHE GUILHOT, CASTELNAUROU (FR); MAMADOU DAFFE, TOULOUSE (FR); CHRISTINE HOUSSIN, CHAVILLE (FR); DAMIEN PORTEVIN, TOULOUSE (FR); CÉLIA DE SOUSA, ORSAY (FR)

Correspondence Address: OBLON, SPIVAK, MCCLELLAND MAIER & NEUSTADT, L.L.P., 1940 DUKE STREET, ALEXANDRIA, VA, 22314, US

(73) Assignees: CENTRE NATIONAL DE LA RECHERCHE SCIENTIFIQUE, PARIS (FR); UNIVERSITE PARIS SUD XI, ORSAY CEDEX (FR)

(21) Appl. No.: 12/240,469

(22) Filed: Sep. 29, 2008

Related U.S. Application Data
(62) Division of application No. 10/570,661, filed on Jan. 3, 2007, now abandoned.

(30) Foreign Application Priority Data
Sep. 4, 2003 (FR) 0310470

(51) Int. Cl. C12N 9/10; C12N 15/63; C12N 5/10; C12N 1/20

(52) U.S. Cl. 435/193; 435/320.1; 435/325; 435/252.1

(57) ABSTRACT

The invention relates to a novel enzyme involved in the biosynthesis of mycolic acids and to the use thereof for the screening of antibiotics, especially antimycobacterials. The invention more particularly relates to the Pks13 protein which catalyzes Claisen condensation or malonic condensation in mycolates between an acyl-CoA molecule or an acyl-AMP molecule and an acylmalonyl-CoA molecule to form an intermediate β-cEto acyl or β-cEto ester.

### CROSS-REFERENCE TO RELATED APPLICATIONS

The present application is a divisional of U.S. Ser. No. 10/570,661, filed on Jan. 3, 2007, which is a National Stage (371) of PCT/FR04/02257, filed on Sep. 6, 2004, which claims priority to FR 0310470, filed Sep. 4, 2003.

The present invention relates to a novel enzyme involved in the biosynthesis of mycolic acids, and to the use thereof in screening for antibiotics, in particular for antimycobacterials.

Mycolic acids are α-alkylated and β-hydroxylated long-chain fatty acids present in the form of esters within the cell walls of bacteria of a specific phylogenetic line of actinomycetes, the suborder Corynebacterineae, also called “mycolata”, comprising the bacterial genera: Mycobacterium, Corynebacterium, Rhodococcus, Nocardia, Gordona and Tsukamurella.

Among the mycolata are major pathogens, in particular the mycobacteria Mycobacterium tuberculosis, the agent for tuberculosis, and Mycobacterium leprae, the agent for leprosy.

For about fifteen years, an upsurge in tuberculosis has been observed, in particular in industrialized countries. This phenomenon is partly linked to the appearance of strains of the tubercular bacillus that are resistant to existing antibiotics. Thus, the design of novel antitubercular medicinal products has again become an important priority.

Among the most effective antitubercular medicinal products are those which interfere with the biosynthesis of the mycobacterial envelope, such as isoniazide, ethionamide and ethambutol (WEBB et al., Molecular Biology and Virulence 1: 287-307 (eds. Ratledge, C, & Dale, J.) (Blackwell Science Ltd, Oxford), 1999). Mycolic acids represent a major constituent of this envelope. It has been reported that they are involved in important biological functions, contributing in particular to bacterial virulence (GLICKMAN et al., Mol. Cell. 5: 717-727, 2000). They are also involved in the low permeability of the envelope of mycolata, which confers on them a natural resistance to many antibiotics (JARLIER et al., J. Bacteriol. 172: 1418-1423, 1990; BRENNAN et al., Annu. Rev. Biochem. 64: 29-63, 1995; DAFE and DRAPER, Adv. Microb. Physiol. 39: 131-203, 1998).

The α- and β-chains of mycolic acids vary in length and in structure (FIG. 1A), but have a common unit (mycolic unit: —CHOH—CHR₂—COOH), which suggests that an enzymatic step involved in the formation of this unit is common to all mycolata.

According to a model that is currently generally accepted (GASTAMBIDE-ODIER et al., Biochemische Zeitschrift 333: 285-295, 1960), the final steps of mycolic acid biosynthesis are thought to consist of a cascade of reactions (FIG. 1B): (1) activation of the acyl so as to form an acyl-CoA molecule, catalyzed by an acyl-CoA synthase; (2) carboxylation of an acyl-CoA molecule so as to form an acylmalonyl-CoA molecule, catalyzed by an acyl-CoA carboxylase; (3) Claisen condensation or malonic condensation of an acyl-CoA or acyl-AMP molecule and of an acylmalonyl-CoA molecule so as to form the β-keto acyl intermediate, catalyzed by a condensase; (4) reduction of the P-keto acyl intermediate so as to form mycolic acid, catalyzed by a reductase.

The mycolic unit would probably be formed during the Claisen condensation or malonic condensation reaction. However, up until now, the enzyme responsible for this condensation had not been identified.

This condensation reaction appears to be similar to the condensation of acyl-CoA with methyl-malonyl-CoA, which occurs in the formation of polymethylated branched fatty acids in mycobacteria (MATHUR et al., J. Biol. Chem. 267: 19388-19395, 1996; SIRAKOVA et al., J. Biol. Chem. 276: 16833-16839, 2001; DUBEY et al., Mol. Microbiol. 45: 1451-1459, 2000), where it is catalyzed by type I polyketide synthases (Pks).

The inventors have put forward the hypothesis that the condensation reaction resulting in mycolic acids in mycolata could be catalyzed by a type I Pks having an unusual substrate specificity.

To verify this hypothesis, they first investigated, using mycolata sequences present in the databases, whether there existed a Pks common to these bacteria and comprising the functional domains required for the condensation reaction, i.e.: an acyltransferase (AT) domain, a ketosynthase domain (KS), an “acyl carrier protein” (ACP) domain, and a thioesterase domain (TE).

They have thus identified, in M. tuberculosis, a gene, called pks13, encoding a type I Pks, and also orthologs of this gene in the other mycobacteria, and also in corynebacteria. These proteins possess high sequence similarities (70 to 80% identity over the entire length of the protein for the various mycobacterial Pks13s and 40 to 50% identity between Pks13 from M. tuberculosis and Pks13 from C. glutamicum or C. diphtheriae), and also possess the domains, mentioned above, which are required for the condensation reaction and for the release of the product.

These proteins will therefore be denoted hereinafter under the general term “Pks13”.

The inventors have also shown that the inactivation of the gene encoding Pks13 results in blocking of the synthesis of mycolic acids, and in a loss of bacterial viability.

Furthermore, they have produced and purified the Pks13 protein in recombinant form.

The results obtained by the inventors show that Pks13 is the condensase involved in mycolic acid synthesis, and that it is a key enzyme in the assembly of the mycolata envelope, and is essential for mycobacterial viability.

A subject of the present invention is a purified protein, called Pks13, involved in mycolic acid biosynthesis and having the following characteristics:

a) it has at least 40% identity, preferably at least 50% identity, and entirely preferably at least 60% identity, over its entire sequence, with the Pks13 protein of M. tuberculosis;
b) it has an acyltransferase domain (pfam00698), a keto acyl synthase domain (pfam02801 or pfam00109), at least one acyl carrier protein domain (COG0331 or COG0304), and a thioesterase domain (COG3319 or pfam00975);
c) it catalyzes a Claisen condensation or malonic condensation between an acyl-CoA or acyl-AMP molecule and an acylmalonyl-CoA molecule.

According to a preferred embodiment of the present invention, said Pks13 protein catalyzes a Claisen condensation between:

a) an acyl-CoA molecule of formula I, or an acyl-AMP molecule of formula Ia:

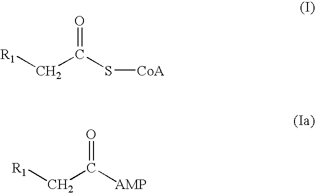

in which R₁ is a chain comprising from 6 to 68 carbon atoms, which may contain one or more —C═C— double bonds, and/or one or more cis/trans-cyclopropane rings,
and/or one or more groups

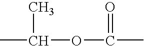

and/or which may carry one or more side groups chosen from —CH₃, ═O and —O—CH₃; and
b) an acylmalonyl-CoA molecule of formula II:

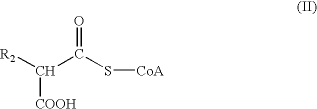

in which R₂ is a linear alkane comprising from 10 to 24 carbon atoms; so as to form a β-keto acyl intermediate of formula III, or a P-keto ester of formula IIIa:

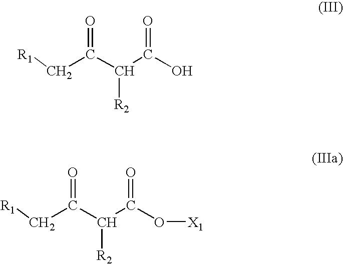

in which R₁ and R₂ are as defined above, and X₁ is an acceptor molecule.

Specific arrangements of this embodiment are as follows:
said Pks13 protein catalyzes the formation of a β-keto acyl of formula III or a β-keto ester of formula IIIa in which R₁ comprises from 6 to 16 carbon atoms and R₂ comprises from 12 to 16 carbon atoms. Said protein can in particular be obtained from the Corynebacterium genus;

said Pks13 protein catalyzes the formation of a β-keto acyl of formula III or of a P-keto ester of formula IIIa in which R₁ comprises from 28 to 48 carbon atoms and R₂ comprises from 14 to 16 carbon atoms. Said protein can in particular be obtained from the Gordona genus;
said Pks13 protein catalyzes the formation of a β-keto acyl of formula III or of a β-keto ester of formula IIIa in which R₁ comprises from 42 to 68 carbon atoms and R₂ comprises from 18 to 24 carbon atoms. Said protein can in particular be obtained from the Mycobacterium genus;
said Pks13 protein catalyzes the formation of a β-keto acyl of formula III or of a β-keto ester of formula IIIa in which R₁ comprises from 24 to 46 carbon atoms and R₂ comprises from 10 to 16 carbon atoms. Said protein can in particular be obtained from the Nocardia genus;
said Pks13 protein catalyzes the formation of a β-keto acyl of formula III or a β-keto ester of formula IIIc in which R₁ comprises from 14 to 34 carbon atoms and R₂ comprises from 10 to 16 carbon atoms. Said protein can in particular be obtained from the Rhodococcus genus;
said Pks13 protein catalyzes the formation of a β-keto acyl of formula III or a P-keto ester of formula IIIa in which R₁ comprises from 40 to 56 carbon atoms and R₂ comprises from 18 to 20 carbon atoms. Said protein can in particular be obtained from the Tsukamurella genus.

According to another preferred embodiment of the present invention, said Pks13 protein has at least 70% identity with the Pks13 protein of M. tuberculosis (SEQ ID NO.: 1).

According to yet another embodiment of the present invention, said Pks13 protein has at least 50% identity, preferably at least 60%, and entirely preferably at least 70% identity, with the Pks13 protein of Corynebacterium glutamicum (SEQ ID NO.: 2).

A subject of the present invention is also an expression vector comprising a polynucleotide sequence encoding a Pks13 protein in accordance with the invention, and also a prokaryotic or eukaryotic, host cell transformed with said expression vector.

A subject of the present invention is also a method for producing a Pks13 protein in accordance with the invention, characterized in that it comprises culturing a host cell in accordance with the invention, and purifying the Pks13 protein from said culture.

A subject of the present invention is also a method for inhibiting the biosynthesis of the mycolata envelope, characterized in that it comprises inhibiting the expression or the activity of the Pks13 protein in said bacteria.

Because it is essential for viability and because of its specificity of action, the Pks13 condensase constitutes an excellent potential target for the design of novel medicinal products, in particular novel antitubercular agents.

Consequently, a subject of the present invention is the use of a Pks13 condensase in accordance with the invention, for screening for antibiotics that are active on mycolata, and in particular on mycobacteria.

The present invention will be understood more clearly from the further description which follows, which refers to examples that illustrate the identification, the production and the purification of the Pks13 condensase, and also the effects of the inactivation thereof on mycolata viability.

### BRIEF DESCRIPTION OF THE DRAWINGS

FIG. 1 shows that mycolic acids vary in length and in structure (FIG. 1A) and provides a model that is currently generally accepted mycolic acid biosynthesis (FIG. 1B).

FIG. 2 shows the pks13 locus in Corynebacterineae.

FIG. 3A shows diagrammatically, the genetic structure of the pks13 locus in the wild-type (WT) strain and in the Δpks13 mutant strain of C. glutamicum. FIG. 3B shows the results of PCR analysis of the Δpks13 mutant and of the wild-type (WT) strain of C. glutamicum as set forth in Example 3. FIG. 3C illustrates the result of the analysis of the fatty acids released after saponification from the wild-type (WT) strain and the Δpks13 mutant and Δpks13:pCGL2308 mutants of C. glutamicum. FIG. 3D shows the freeze-fracture plane of wild-type (WT) strain and the Δpks13 mutant of C. glutamicum.

FIG. 4A shows diagrammatically, the genetic structure of the pks13 locus obtained during the construction of the PMM48:pDP32 conditional mutant of M. smegmatis. FIG. 4B shows the results of PCR analysis of the PMM48:pDP32 conditional mutant of M. smegmatis and of its parental strains PMM47 and mc2155 (WT). FIG. 4C shows the growth curves at 30° C. and 42° C. of the PMM48:pDP32 conditional mutant of M. smegmatis and of its parental mc2155 (WT) strain. FIG. 4D shows the analysis of the fatty acids released after saponification from the wild-type strain of M. smegmatis and from the conditional mutant PMM48:pDP32, after growth at a permissive temperature (30° C.) or nonpermissive temperature (42° C.).

FIG. 5 shows two reaction sequences of producing mycolate from difference substrates.

FIG. 6 shows differences in colony morphology for the wild-type C. glutamicum, Δpks13::km mutant strain of C. glutamicum, ΔfadD32::km mutant, and ΔaccD4::km mutant.

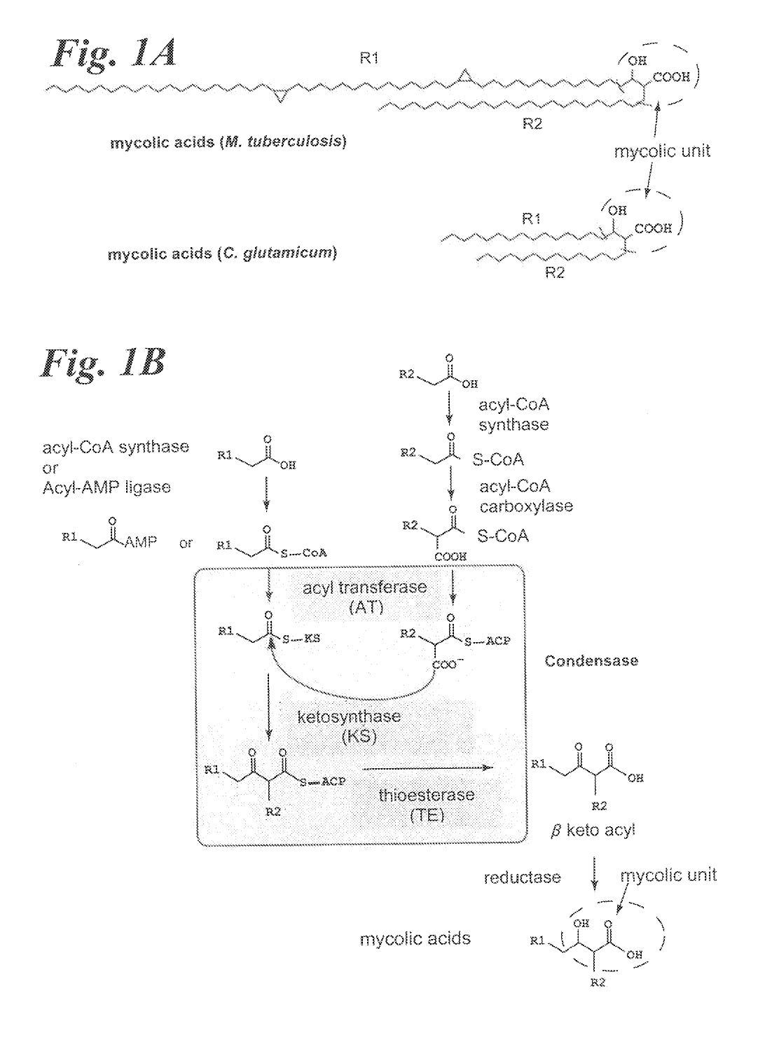

### EXAMPLE 1

### Identification of the PKS13 Condensase

M. tuberculosis contains 16 type I Pks enzymes, among which 9 are also found in M. leprae. Among these 9 putative enzymes, 7 are already known to be involved in the biosynthesis of other lipid groups in M. tuberculosis (AZAD et al., J. Biol. Chem. 272: 16741-16745, 1997; CONSTANT et al., J. Biol. Chem. 277: 30 38148-38158, 2002). Among the remaining two candidate proteins, the one called ML1229 has the same domain organization as and also strong sequence similarities with the type I Pks enzymes of M. tuberculosis that are involved in the biosynthesis of branched polymethyl fatty acids. The second candidate is called Pks13 in M. tuberculosis and MLO101 in M. leprae.

Analysis of the deduced sequence of Pks13 (accession number NP_{—}338459; 1733 amino acids) of M. tuberculosis CDC1551 reveals the presence of the various catalytic domains required and sufficient for the catalysis of the Claisen condensation involved in mycolic acid formation: two “acyl carrier protein” (ACP) domains (amino acids 39 to 107 and 1237 to 1287), a “ketosynthase” (KS) domain (amino acids 119 to 543), an “acyltransferase” (AT) domain (amino acids 640 to 1045), and a “thioesterase” (TE) domain (amino acids 1464 to 1543).

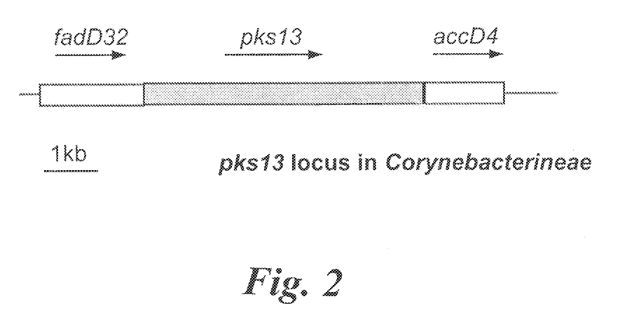

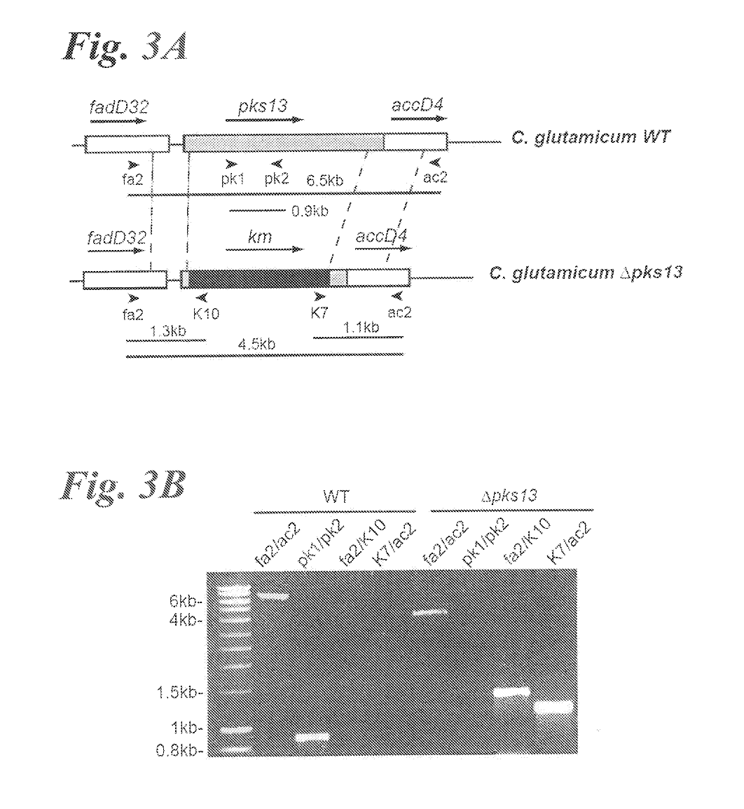

Orthologs of ML1229 and Pks13 have been sought in various species using the BLAST program (ALTSCHUL et al., Nucleic Acid Res. 25: 3389-3402, 1997). The sequences of various putative Pks13 condensases encoded by the pks13 gene, “acyl-CoA synthase” FadD32 and “acyl-CoA carboxylase subunit” AccD4 (encoded, respectively, by two genes, fadD32 and accD4, flanking the psk13 gene in all the corynebacteria and mycobacteria analyzed, as illustrated in FIG. 2) were compared using the Needleman-Wunsch program available on the website of the Pasteur Institute http://www.pasteur.fr.

No ML1229 ortholog was identified in three species of corynebacteria (C. glutamicum, C. efficiens and C. diphtheriae), whereas Pks13 orthologs (ML0101) were found in the three species of corynebacteria mentioned above and in three other species, of mycobacteria (M. smegmatis, M. marinum and M. avium). These Pks13 proteins contain the catalytic domains required for the condensation resulting in mycolic acid synthesis, and the corresponding genes are located downstream of genes known to be involved in the transfer of mycolic acid to arabinogalactan (PUECH et al., Mol. Microbiol. 44: 1109-1122, 2002). The sequence identities of the Pks13 proteins, relative to the complete sequence of Pks13 of M. tuberculosis, are given in table 1 below:

TABLE 1

| M. tuberculosis | M. leprae | M. smegmatis | M. marinum | M. avium | C. glutamicum | C. efficiens | C. diphtheriae |
|---|---|---|---|---|---|---|---|
| FadD32 | 93% | 75% | 93% | 83% | 40% | 42% | 42% |
| Pks13 | 83% | 71% | 84% | 81% | 44% | 43% | 44% |
| AccD4 | 91% | 81% | 85% | 80% | 54% | 52% | 53% |

The presence of Pks13 was also demonstrated in other mycolic acid-producing bacterial species, by PCR-amplifying a 1 kb internal fragment of pks13 from the genome of, Nocardia asteroides ATCC19243, Rhodococcus rhodochrous ATCC13808 and Tsukamurella paurometabolum CIP100753T, using the following degenerate primers: pks13a: 5′-GCTGGARCTVACVTGGGARGC-3′ (SEQ ID NO.: 3) pks13b: 5′-GTGSGCGTTGGYDCCRAAVCCGAA-3′ (SEQ ID NO.: 4).

The PCR conditions are: 2.5 units of Taq polymerase (Roche Molecular Biochemicals), 10% of dimethyl sulfoxide (Me₂SO), 1 mM of dNTP and 4 μM of each primer in a final volume of 50 μl, under the conditions recommended by the supplier (Roche Molecular Biochemicals). The amplification program is: 5 min at 94° C., then 35 cycles of 1 min at 94° C., 1 min at 58° C., 1 min 30 sec at 72° C., then 1 cycle of 10 minutes at 72° C. For T. paurometabolum, the steps at 58° C. are replaced with steps at 50° C.

The sequences of these fragments exhibit 40% identity over their entire length with Pks13 of M. tuberculosis, also suggesting the presence of pks13 in these bacteria.

All these results suggest that the Pks13 protein is found in all mycolic acid-producing mycolata, and that, among the type I Pks enzymes, Pks13 is the only enzyme capable of catalyzing the condensation of the α- and β-chains of fatty acids so as to form mycolic acids.

### EXAMPLE 2

### Cloning, Overexpression and Purification of the Pks13 Proteins of Mycobacterium Tuberculosis and Corynebacterium glutamicum

### Plasmid Construction

The C. glutamicum ATCC13032 strain (DUSCH et al., Appl. Environ. Microbiol. 65: 1530-1539, 1999) is cultured on a BHI medium (DIFCO). The M. tuberculosis H37Rv strain is cultured on a Middlebrook 7H9 liquid medium (DIFCO) supplemented with 10% ADC (DIFCO) and with 0.05% Tween 80.

The culture media are supplemented with kanamycin, hygromycin, chloramphenicol and sucrose when necessary at a final concentration of 40 μg/ml, 50 μg/ml, 15 μg/ml and 10% (w/v), respectively.

The total bacterial DNA is extracted from 5 ml of saturated liquid cultures as described in Belisle et al., 1998. The DNA pellets are resuspended in 100 μl of 10 mM Tris (pH 8).

Plasmids pWM35 and pWM36

The pks13 gene of M. tuberculosis is amplified by PCR from the total DNA of the H37Rv strain and using the primers 13Rtb 5′-GAGGACATATGGCTGACGTAGCGGAATC-3′ (SEQ ID NO.: 5) and 13Stb 5′-CGGTGAAAGCTTCTGCTTGCCTACCTCACTTG-3′ (SEQ ID NO.: 6), with 2.5 units of Pfu DNA polymerase (Promega, Lyons, France), 10% of dimethyl sulfoxide (Me₂SO), and 1 μM of each primer in a final volume of 50 μl, under the conditions recommended by the supplier (Promega, Lyons, France). The amplification program is: 5 min at 94° C., then 30 cycles of 1 min at 94° C., 1 min at 57° C., 5 min at 72° C., then 10 min at 72° C. The amplification product is purified using the Qiaquick kit (Qiagen, Courtaboeuf, France), and then digested with the NdeI/HindIII restriction enzymes. The fragment obtained is inserted into the vector pET26b (Novagen), itself cleaved with the NdeI/HindIII restriction enzymes. The resulting plasmid, called pWM35, contains the pks13 gene fused in the position 3′ of the gene comprising a tag formed from 18 nucleotides encoding a sequence of 6 histidines.

The pks13 gene of M. tuberculosis is amplified by PCR from the total DNA of the H37Rv strain and using the primers 13Rtb 5′-GAGGACATATGGCTGACGTAGCGGAATC-3′ (SEQ ID NO.: 5) and 13Ttb 5′-GCTCGGGGATCCTCACTGCTTGCCTACCTCAC-3′ (SEQ ID NO.: 7), under the same conditions as those described above. The amplification product is purified as described above, and then digested with the NdeI/BamHI restriction enzymes. The fragment obtained is inserted into the vector pET15b (Novagen) cleaved beforehand with the NdeI/BamHI restriction enzymes. The resulting plasmid, pWM36, possesses the pks13 gene fused in the position 5′ of the gene comprising a tag of 18 nucleotides encoding a sequence of 6 histidines.

Plasmid pWM38

The pks13 gene of C. glutamicum ATCC13032 is amplified by PCR from the total DNA of this strain and using the primers 13Ccg 5′-AATATGACTAGTAGCCAATCGTCGGATCAGAAG-3′ (SEQ ID NO.: 8) and 13Dcg 5′-AGCTCTAGATCTCTAATTCTTCCGAGAAATCTCAT-3′ (SEQ ID NO.: 9), under the same conditions as those described above. The amplification product is purified as above, and then digested with the SpeI/BglII restriction enzymes. The fragment obtained is inserted into the vector pET15b that has been modified by the insertion of an SpeI site in place of the XhoI site, and then cleaved with the SpeI/BamHI restriction enzymes. The resulting plasmid, pWM38, possesses the pks13 gene of C. glutamicum, fused to a tag of 18 nucleotides, that is in the 5′ position, of the gene encoding a sequence of 6 histidines.

Overexpression of the Pks13 Proteins of Mycobacterium tuberculosis and Corynebacterium glutamicum in Escherichia coli

The plasmids pWM35, pWM36 and pWM38 are transferred into the Escherichia coli strain BL21 (DE3): pLysS (Novagen).

The three strains are inoculated into 3 ml of LB medium containing chloramphenicol (30 μg/ml) and kanamycin (40 μg/ml) or ampicillin (100 μg/ml) depending on the plasmids. The cultures are incubated at 37° C. with shaking (250 rpm) until saturation.

A 1/100^{th} dilution of these cultures is prepared in 200 ml of LB medium containing kanamycin or ampicillin. These new cultures are incubated with shaking at 37° C. for 2 h 30 min (OD₆₀₀ₙₘ=0.7-0.8). Isopropylthio-β-D-galactoside (IPTG) is added at a final concentration of 0.5 mM and the culture is incubated for 3 h at 30° C. with shaking.

Purification of the Pks13 Proteins of Mycobacterium tuberculosis and Corynebacterium glutamicum

The cells expressing the various Pks13 proteins are pelleted by centrifugation at 2500 g for 15 min, and then taken up in 40 ml of loading buffer (50 mM Tris-HCl, pH=7.5, 5 mM imidazole, 300 mM NaCl). The cells are frozen at −20° C. for 15 h, and are then subjected to 3 cycles of thawing-freezing in liquid nitrogen. They are then sonicated 3 times for 30 sec (Vibra-cell, Bioblock Scientific) (50% active cycle and output power 5), and then centrifuged for 30 min at 20 000 g.

The supernatant is filtered through a microfilter (pore diameter: 0.2 μm) and then loaded onto a “Chelating Sepharose Fast Flow” column (Amersham) in FPLC (Biorad HP duoflow). The protein is eluted by means of a gradient of 5 to 150 mM of imidazole with an elution peak at 90 mM. The protein-enriched fractions are mixed, concentrated by filtration on a centriprep 30 (Amicon), and the protein is separated from the residual contaminants by exclusion chromatography (S-200 16/60 mm, Amersham) in FPLC.

By following this procedure, approximately 20 mg of Pks13 proteins of M. tuberculosis or of C. glutamicum are obtained.

### EXAMPLE 3

### Biochemical Analysis of Δpks13 Mutants of Corynebacterium glutamicum and Mycobacterium smegmatis

The C. glutamicum ATCC13032 strain is cultured as described above.

The wild-type M. smegmatis mc²155 strain (SNAPPER et al., Mol. Microbiol. 4: 1911-1919, 1990) is cultured on an LB medium (Difco) supplemented with 0.05% of Tween 80 in order to prevent aggregation.

The culture media are supplemented with kanamycin, hygromycin, chloramphenicol and sucrose when necessary at a final concentration of 40 μg/ml, 50 μg/ml, 15 μg/ml and 10% (w/v), respectively.

The total bacterial DNA is extracted from 5 ml of saturated liquid culture as described in Belisle et al., 1998. The DNA pellet is resuspended in 100 μl of 10 mM Tris (pH 8).

Construction of a C. glutamicum Mutant
Δpks13 Mutant Strain of C. glutamicum

Two DNA fragments of 0.9 kb and 0.7 kb overlapping the pks13 gene on its 5′ and 3′ ends are amplified by PCR from the total DNA of C. glutamicum using, respectively, the following pairs of primers:

(SEQ ID NO.: 10)

| | |
|---|---|
| pkde15: | |
| 5′-GAAATCTCGAGCCACGGCGAAA-3′ | (Tm = 54° C.) |

(SEQ ID NO.: 11)

| | |
|---|---|
| pkde12: | |
| 5′-ACGATTGCCGCGGTTCCATATTG-3′ | (Tm = 54° C.) |
| and | |

(SEQ ID NO.: 12)

| | |
|---|---|
| pkde13: | |
| 5′-CATCCTGTTCCGCGGAACGCATGC-3′ | (Tm = 54° C.) |

(SEQ ID NO.: 13)

| | |
|---|---|
| pkde14: | |
| 5′-CAGCATGATGGAGATCTGAGGGC-3′ | (Tm = 54° C.). |

The PCR conditions are: 1 unit of Taq polymerase (Roche Molecular Biochemicals), 2 mM MgCl₂, 0.2 mM of dNTP and 0.5 μM of each primer in a final volume of 50 μl, under the conditions recommended by the supplier (Roche Molecular Biochemicals). The amplification program is: 2 min at 94° C., then 35 cycles of 1 min at 94° C., 30 sec at 54° C., 1 min 30 sec at 72° C., then 1 cycle of 10 min at 72° C.

These fragments are inserted into the plasmid pMCSS (Mobitec, Gottingen, Germany). A kanamycin-resistance cassette is inserted between these two PCR fragments, to give the plasmid pCMS5::pks. This plasmid is transferred into the C. glutamicum strain and the transformants are selected on an agar medium containing kanamycin.

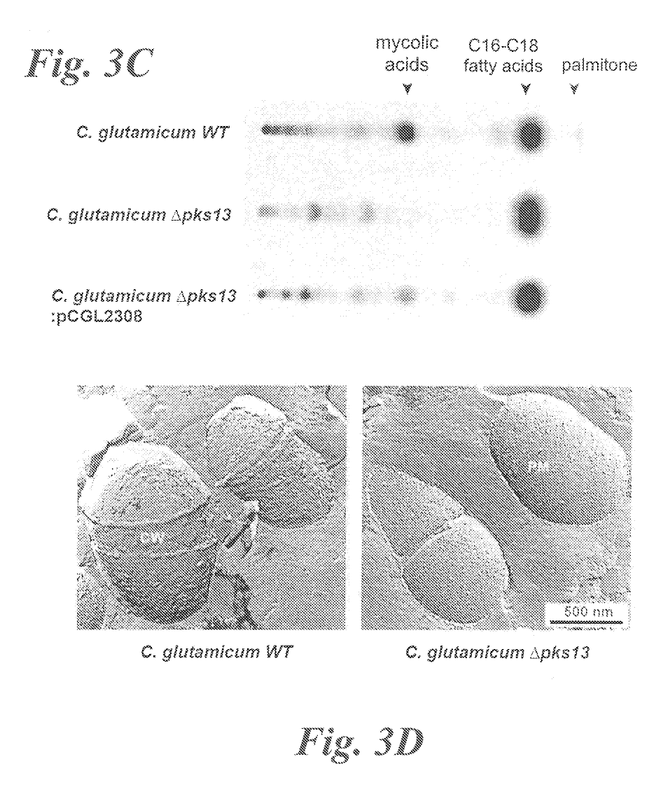

FIG. 3A shows, diagrammatically, the genetic structure of the pks13 locus in the wild-type (WT) strain and in the Δpks13 mutant strain of C. glutamicum. In the latter, the wild-type pks13 allele present on the chromosome is replaced with a mutated allele containing an internal deletion of 4.3 kb into which the km gene encoding kanamycin is inserted. The boxes indicate the various genes of the pks13 locus. The location and the name of the primers used for the PCR analysis of the mutant strains are indicated by arrowheads. The PCR amplification products expected for the various strains are indicated under each genetic structure.

The Δpks13 transformants in which the allelic replacement has occurred between the wild-type chromosomal pks13 gene and the mutated plasmid allele exhibit (1) a change in colony morphology, from a shiny smooth appearance to a rough appearance, (2) a considerably decreased growth curve (doubling of division time) compared with the wild type, (3) a thermosensitivity which makes them incapable of growing at temperatures above 30° C., unlike the wild type which produces colonies on agar medium up to 37° C., and (4) a high degree of aggregation in liquid culture in the absence of detergent.

These transformants are characterized by PCR using the following primers:

(SEQ ID NO.: 14)

fa2:
5′-TCTGACCACCTTCCGTGAAGC-3′
(Tm = 55° C. or 62° C.)

(SEQ ID No.: 15)

ac2:
5′-GAACGAGTTCAGAGCTTC-3′
(Tm = 55° C. or 62° C.)

(SEQ ID No.: 16)

K10:
5′-TATTTCGAATGGTTCGCTGGGTTTATC-3′
(Tm = 55° C.)

(SEQ ID No.: 17)

K7:
5′-TAAAAAGCTTATCGATACCG-3′
(Tm = 55° C.)

(SEQ ID No.: 18)

pk1:
5′-GCCGTGACGGTATCTCGG-3′
(Tm = 55° C.)

(SEQ ID No.: 19)

pk2:
5′-CCAGGGCAGTTGCTTCAATG-3′
(Tm = 55° C.).

FIG. 3B gives the results of PCR analysis of the Δpks13 mutant and of the wild-type (WT) strain of C. glutamicum.

Δpks13:pCGL2308 Mutant Strain of C. glutamicum

A complementation plasmid, pCGL2308, is produced by the insertion into the vector pCGL482 (PEYRET et al., Mol. Microbiol. 9: 97-109, 1993) of a 5.3 kb fragment from C. glutamicum, comprising the pks13 gene and the 417 by region upstream of this gene, obtained by PCR from the total DNA of C. glutamicum using the following pair of primers:

(SEQ ID No.: 20)

| | | |
|---|---|---|
| pk3: | 5′-TCCGGAAAGATCTCACGCCGCG-3′ | (Tm = 62° C.) |

(SEQ ID No.: 21)

| | | |
|---|---|---|
| pk4: | 5′-GCGTGCGCGCAGATCTGCTAGC-3′ | (Tm = 62° C.). |

The resulting plasmid pCGL2308 is transferred by electroporation into the Δpks13 strain of C. glutamicum and the Δpks13: pCGL2308 transformants are selected on agar medium containing kanamycin.

The Δpks13:pCGL2308 transformants exhibit a shiny and smooth morphology, an intermediate growth rate between the wild-type strain and the mutant strain, an inability to grow at temperatures above 32° C. (whereas the wild-type strain grows at 37° C.), and a mycolic acid content that is much lower than that of the wild-type strain.

It therefore appears that the complementation with the plasmid induces a partial reversion to the wild-type phenotype.

Construction of a Conditional Mutant of M. smegmatis PMM47 Mutant Strain of M. smegmatis

Two DNA fragments of approximately 1 kb overlapping the pks13 gene on its 5′ and 3′ ends are amplified by PCR from the total DNA of M. smegmatis using, respectively, the following pairs of primers:

(SEQ ID No.: 22)

| | |
|---|---|
| 13F: | 5′-GCTCTAGAGTTTAAACGCTGGACCTGTCCAACGTCAAGG-3′ |

(SEQ ID No.: 23)

| | |
|---|---|
| 13G: | 5′-GGACTAGTCGTCGAAACCGACCGTCACCAG-3′ |
| and | |

(SEQ ID No.: 24)

| | |
|---|---|
| 13H: | 5′-GGACTAGTCGGCATCTTCAACGAGTTGC-3′ |

(SEQ ID No.: 25)

| | |
|---|---|
| 13I: | 5′-CCCAAGCTTGTTTAAACTTGTCGAAGTGGTTCGACGG-3′. |

The PCR conditions are: 3 units of Pfu polymerase (Promega, Lyons, France), 10% of dimethyl sulfoxide (Me₂SO), 1 mM of dNTP and 1 μM of each primer in a final volume of 50 μl, under the conditions recommended by the supplier (Promega, Lyons, France).

The amplification program is: 5 min at 94° C., then 30 cycles of 1 min at 94° C., 1 min at 58° C., 3 min at 72° C., then 1 cycle of 10 min at 72° C.

These fragments are inserted into the plasmid pJQ200 (QUANDT et al., Gene 127: 15-21, 1993). A hygromycin-resistance cassette is inserted between these two PCR fragments, to give the plasmid pDP28. This nonreplicative plasmid containing the sacB marker and a copy of the mutated allele pks13::hyg is transferred into the M. smegmatis strain by electroporation and the transformants are selected on agar medium containing hygromycin.

The transformants that have integrated the plasmid pDP28 by simple recombination between the copies of the wild-type pks13 gene and the mutated pks13 gene are characterized by PCR using the following primers:

| | | |
|---|---|---|
| 13J: | 5′-CTTCCACGACATGGTCTGAT-3′ | (SEQ ID No.: 26) |
| 13K: | 5′-CACGATCGAGTCGAGCTCGA-3′ | (SEQ ID No.: 27) |
| H1: | 5′-AGCACCAGCGGTTCGCCGT-3′ | (SEQ ID No.: 28) |
| H2: | 5′-TGCACGACTTCGAGGTGTTCG-3′. | (SEQ ID No.: 29) |

The PCR conditions are: 2.5 units of Tag polymerase (Roche Molecular Biochemicals), 10% of dimethyl sulfoxide (Me₂SO), 1 mM of dNTP and 1 μM of each primer in a final volume of 50 μl, under the conditions recommended by the supplier (Roche Molecular Biochemicals). The amplification program is: 5 min at 94° C., then 30 cycles of 1 min at 94° C., 1 min at 62° C., 2 min 30 sec at 72° C., then 1 cycle of 10 min at 72° C. An M. smegmatis strain called PMM47 is selected, in which the plasmid pDP28 is inserted at the pks13 locus by simple recombination. Plating out a culture of PMM47, at various temperatures (25° C., 32° C. or 37° C.), on a medium containing 10% of sucrose and hygromycin produces clones with a mutation in the sacB gene, but no second recombination event that can produce a strain carrying only the mutated allele pks13::hyg is selected.

This result indicates that the pks13 gene is essential for mycobacterial growth. In order to confirm this hypothesis, a second copy of the wild-type pks13 gene is transferred into PMM47 cloned on a thermosensitive mycobacterial vector.

PMM48:pDP32 Thermosensitive Mutant Strain of M. smegmatis

In order to produce the complementation plasmid pDP32, the pks13 gene is amplified by PCR from the total DNA of M. smegmatis using the primers 13R 5′-ATGAGATCTGATGAAAACCACAGCGAT-3′ (SEQ ID No.: 30) and 13P 5′-GGACTAGTCTTGGCGACGGCCTTCTCAC-3′ (SEQ ID No.: 31).

The PCR conditions are: 3 units of Pfu DNA polymerase (Promega, Lyons, France), 10% of dimethyl sulfoxide (Me₂SO), 1 mM of dNTP, and 1 μM of each primer in a final volume of 50 μl, under the conditions recommended by the supplier (Promega, Lyons, France). The amplification program is: 5 min at 94° C., then 30 cycles of 1 min at 94° C., 1 min at 58° C., 5 min at 72° C., then 10 min at 72° C.

The pks13 gene is inserted into a thermosensitive mycobacterial plasmid derived from the plasmid pCG63 (GUILHOT et al., FEMS Microbiol. Letter 98: 181-186, 1992) and containing a mycobacterial expression cassette, with a mycobacterial promoter, pBlaF*, upstream of a multiple cloning site, itself upstream of a transcription terminator (LE DANTEC et al., J. Bacteriol. 183: 2157-2164, 2001). The resulting plasmid pDP32 is transferred by electroporation into the PMM47 strain of M. smegmatis and the transformants are selected on agar medium containing kanamycin and hygromycin. The second recombination at the pks13 chromosomal locus is selected by plating out a liquid culture of these transformants at 30° C. on agar medium containing kanamycin, hygromycin and sucrose at 30° C. The colonies are screened by PCR using the following primers:

| | | |
|---|---|---|
| 13J: | 5′-CTTCCACGACATGGTCTGAT-3′ | (SEQ ID No.: 26) |
| 13K: | 5′-CACGATCGAGTCGAGCTCGA-3′ | (SEQ ID No.: 27) |
| H1: | 5′-AGCACCAGCGGTTCGCCGT-3′ | (SEQ ID No.: 28) |
| H2: | 5′-TGCACGACTTCGAGGTGTTCG-3′. | (SEQ ID No.: 29) |

The PCR conditions are: 2.5 units of Taq polymerase (Roche Molecular Biochemicals), 10% of dimethyl sulfoxide (Me₂SO), 1 mM of dNTP and 1 μM of each primer in a final volume of 50 μl, under the conditions recommended by the supplier (Roche Molecular Biochemicals). The amplification program is: 5 min at 94° C., then 30 cycles of 1 min at 94° C., 1 min at 62° C., 2 min 30 sec at 72° C., then 1 cycle of 10 min at 72° C.

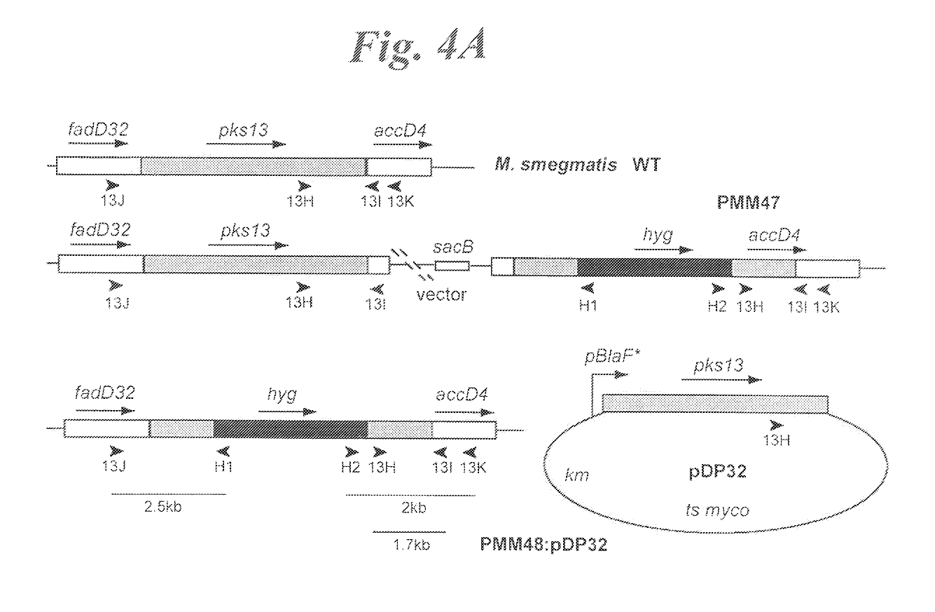

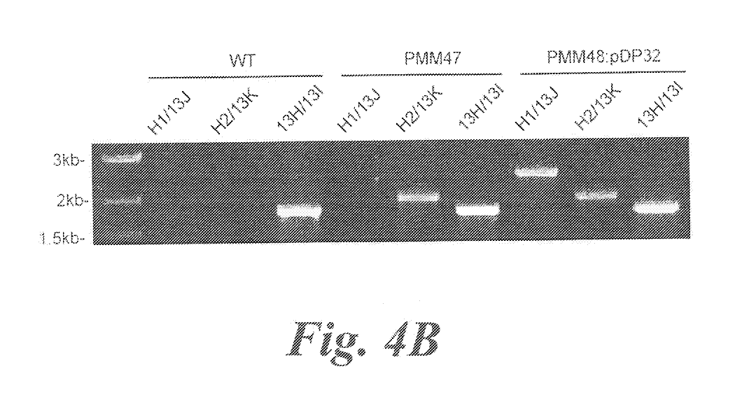

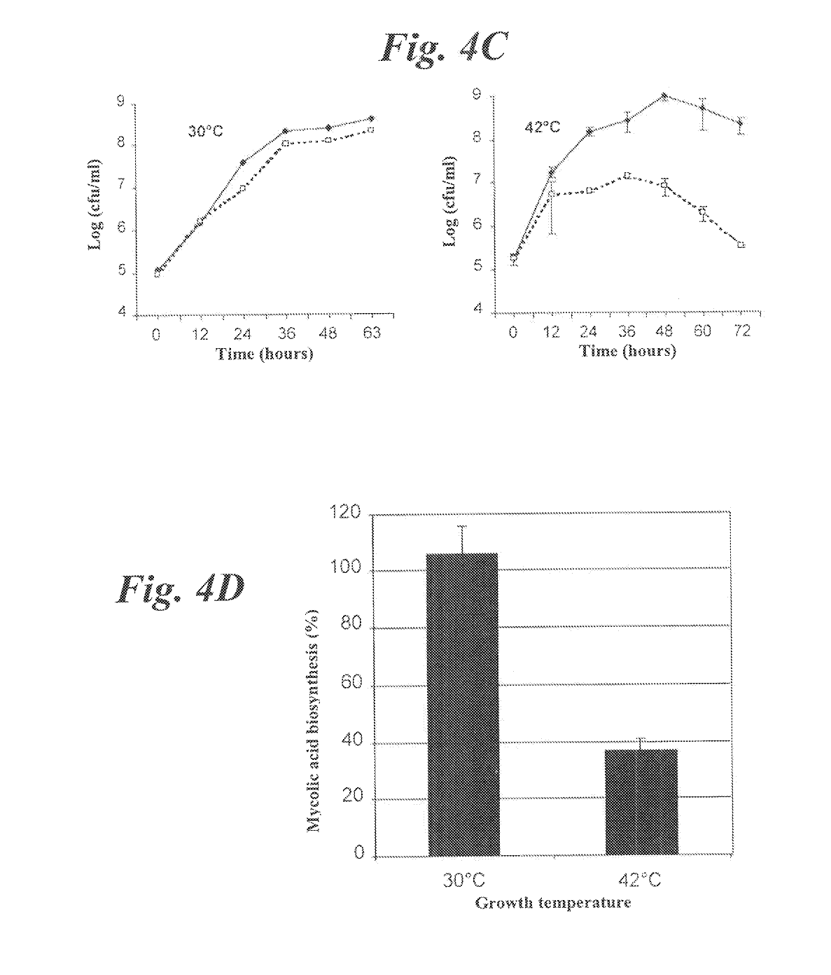

FIG. 4A shows, diagrammatically, the genetic structure of the pks13 locus obtained during the construction of the PMM48:pDP32 conditional mutant of M. smegmatis. The boxes indicate the various genes of the pks13 locus. The location and the name of the primers used for the PCR analysis of the mutant strains are indicated by arrowheads. The PCR amplification products expected for the various strains are indicated under each genetic structure.

FIG. 4B shows the results of PCR analysis of the PMM48:pDP32 conditional mutant of M. smegmatis and of its parental strains PMM47 and mc²155 (WT).

Using these conditions, 8% of the Hyg^{R}, Km^{R}, Suc^{R} colonies selected are the result of an allelic exchange; the other clones being the result of a mutation of the sacB gene.

The strain called PMM48:pDP32, in which the wild-type chromosomal copy of the pks13 gene is replaced with the pks::hyg mutated allele and a functional copy of the pks13 gene is on a thermosensitive plasmid, is selected for a phenotypic analysis. The results are represented in FIG. 4C.

Legend of FIG. 4C:

□=recombinant strain PMM48:pDP32 of M. smegmatis
♦=wild-type (WT) strain.

Plating out this recombinant strain on agar medium containing hygromycin at 32° C. or 42° C. reveals that it is incapable of forming colonies at high temperature. In liquid culture at 32° C., this strain grows as quickly as the wild-type strain, this temperature being a temperature that is permissive for the plasmid pDP32. However, when the culture is placed at 42° C., which is a temperature that is nonpermissive for the plasmid pDP32, the number of viable bacteria increases up to the time 12 h to 24 h post-inoculation, then remains stable over the next 24 hours, before decreasing; the only viable bacteria are those which have conserved a copy of the complementation plasmid.

These results show that the pks13 gene is essential for the survival of M. smegmatis, as expected of a gene encoding an enzyme involved in mycolic acid biosynthesis.

Biochemical Analysis of the Δpks13 Mutant of C. glutamicum and the PMM48:pDP32 Mutant of M. smegmatis

### Analytical Protocol

The C. glutamicum strains are cultured up to the exponential phase and labeled with 0.5 μCi/ml of [¹⁴C]acetate (specific activity of 54 mCi/mmol; ICN, Orsay, France) for 3 h. For the radiolabeling of the conditional mutant of M. smegmatis at nonpermissive temperature, PMM48:pDP32 and the wild-type strain mc²155 are cultured at 30° C. These cultures are then diluted in fresh medium at an OD₆₀₀ₙₘ=0.005 and incubated at 42° C. until an OD₆₀₀ₙₘ=0.3 is reached. The cells are then labeled for 3 h with 0.5 μCi/ml [¹⁴C]acetate.

The fatty acids are prepared from the labeled cells and separated by thin layer chromatography on Durasil 25 using dichloromethane or an ether/diethyl:ether (9:1) mixture as eluant as described in Laval et al. (Anal. Chem. 73: 4537-4544, 2001). The labeled compounds are quantified on a Phosphorimager (Amersham Biosciences).

For the analyses by gas chromatography followed by mass spectrometry analysis (GC-MS), trimethylsilyl derivatives of fatty acids are obtained as described in Constant et al. (J. Biol. Chem. 277: 38148-38158, 2002) and analyzed on a Hewlett-Packard 5889X mass spectrometer (electron energy, 70 eV) working in electron-capture (EI) modes using NH₃ as reaction gas (Cl/NH₃), coupled with a Hewlett-Packard 5890 series II gas chromatograph combined with a similar OV1 column (0.30 mm×12 m).

### Results

Δpks13 and Δpks13:pCGL2308 mutants of C. glutamicum

FIG. 3C illustrates the result of the analysis of the fatty acids released after saponification from the wild-type (WT) strain and the Δpks13 and Δpks13:pCGL2308 mutants of C. glutamicum. The thin layer chromatography analysis of these products reveals that the spots corresponding to the mycolic acids or to palmitone, a product of degradation of the β-keto acyl intermediate resulting from the condensation reaction, are no longer detectable in the mutants. This observation is confirmed by the GC-MS analysis, which demonstrates that the Δpks13 mutant of C. glutamicum no longer synthesizes any mycolic acids but produces an amount of C16-C18 fatty acids, the precursor of mycolate, that is similar to that of the wild-type strain (data not shown). This mycolic acid production is partially restored following the transfer into the Δpks13 mutant strain of a plasmid carrying the functional pks13 gene of C. glutamicum; which demonstrates that these phenotypes are effectively due to the deletion of pks13. The partial restoration suggests either that the expression of pks13 by the plasmid is not of the same level as that in the wild-type strain, or that the chromosomal insertion of the kanamycin cassette exerts a polar effect on the expression of the accD4 gene, or both.

Furthermore, in the Mycolata, mycolic acids are supposed to contribute to the lipid bilayer which forms a functional homologue of the outer membrane of Gram-negative bacteria. In corynebacteria and mycobacteria, a freeze-fracture plane is propagated between the two layers of this outer pseudomembrane. As expected, FIG. 3D shows the loss of this fracture plane in the Δpks13 mutant strain of C. glutamicum, whereas it is clearly visible in the wild-type strain, which suggests that the lipid bilayer composed predominantly of mycolic acids is no longer present in the mutant.

These results demonstrate that the Δpks13 mutant of C. glutamicum is clearly depleted of an enzyme that is essential in mycolic acid biosynthesis.

PMM48:pDP32 Mutant of M. smegmatis

FIG. 4D illustrates the result of the analysis of the fatty acids released after saponification from the wild-type strain of M. smegmatis and from the conditional mutant PMM48:pDP32, after growth at a permissive temperature (30° C.) or nonpermissive temperature (42° C.). The mycolate/short-chain fatty acid ratio is quantified for the PMM48:pDP32 mutant and divided by that obtained for the wild-type strain cultured under the same conditions. The graph shows that, after transfer to 42° C., the average mycolate content in the PMM48:pDP32 mutant is decreased by more than 60%. As expected, this synthesis is not completely stopped in the culture because the remaining bacterial population conserving the nonreplicative complementation plasmid produces mycolic acids.

These results show that the pks13 gene is involved in mycolic acid biosynthesis in M. smegmatis.

### EXAMPLE 4

### Screening for Antibiotics that are Active on Mycolata

Screening for Xenobiotics that Inhibit the Condensation by Pks13; Directly or Indirectly

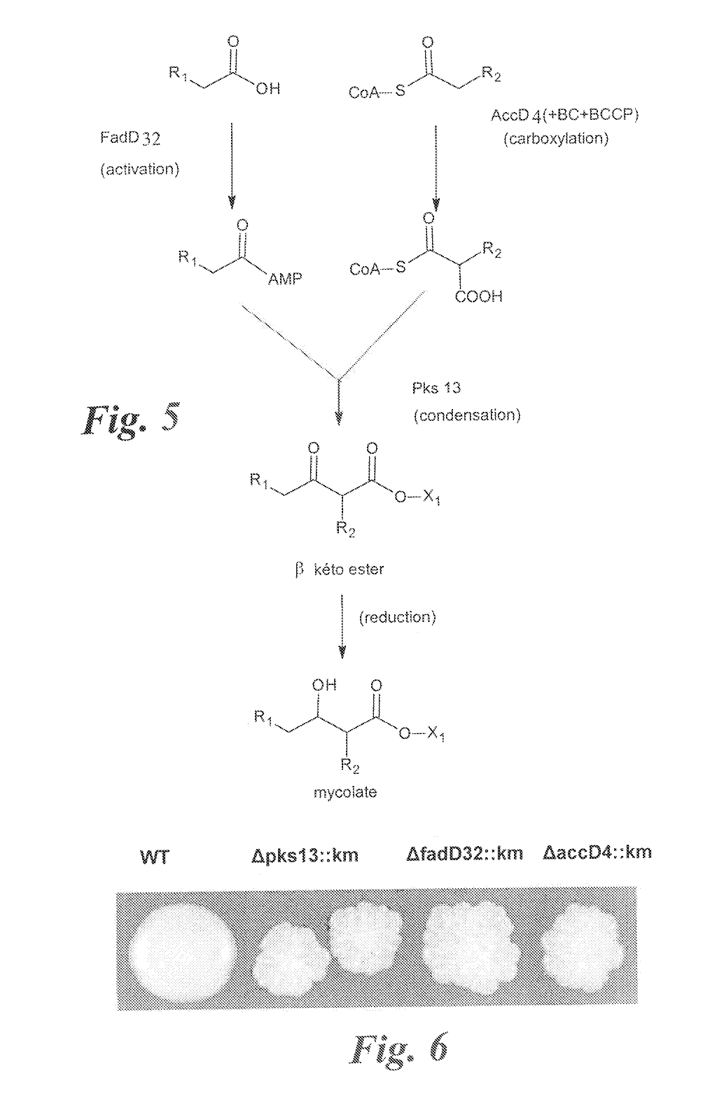

As illustrated in FIG. 5, Pks13 allows the condensation of two substrates, which themselves result from two independent reactions.

The absence of mycolic acids in mycolata can therefore come from the inhibition of Pks13 and/or from the inhibition of FadD32, and/or from the inhibition of the carboxylase complex in which the AccD4 protein is involved.

Several tests make it possible to screen for the action of a xenobiotic on mycolic acid synthesis by mycolata.

As seen in example 3 above, the Δpks13 transformants in which the pks13 gene has been inactivated show a change in the colony morphology, which goes from a shiny smooth appearance to a rough appearance. This is also the case for C. glutamicum bacteria in which the accD4 or fadD32 gene is mutated (see FIG. 6). A first test to determine the impact of a xenobiotic on mycolic acid synthesis therefore consists in plating out mycolata capable of surviving without producing mycolic acids, for example C. glutamicum bacteria (for example, the ATCC13032 strain), on an agar culture medium containing the xenobiotic to be tested. Visual observation of the colonies obtained makes it possible to identify the potential antibiotics.

Another test consists in growing C. glutamicum bacteria in liquid medium, as described above, in the presence or in the absence of the xenobiotic to be tested. 0.5 μCi/ml [¹⁴C]acetate (specific activity of 54 mCi/mmol; ICN, Orsay, France) is added during the exponential growth phase, for at least 3 hours, before carrying out the biochemical analysis of the fatty acids contained in the bacteria by thin layer chromatography, as described above and in Portevin et al., PNAS 2004, Vol. 101, p 314-319 (see in particular the first paragraph of page 316). As illustrated in FIG. 3C, it is possible to detect the mycolic acids synthesized by the strain cultured in the absence of the xenobiotic (control), and also palmitone, a degradation product resulting from the condensation reaction with Pks13. An impairment of the function of Pks13, and/or of FadD32, and/or of the carboxylase complex, related to the presence of the xenobiotic, will lead to a decrease, or seven the disappearance, of the corresponding bands.

Of course, a xenobiotic identified according to one of the two tests described above can subsequently be tested for its ability to inhibit the growth of mycolata incapable of surviving without producing mycolic acids, such as Mycobacterium tuberculosis and Mycobacterium leprae.

Determination of the Step of Mycolic Acid Synthesis that is Effectively Inhibited by the Xenobiotic

A second analytical step is necessary in order to determine more finely the target of a xenobiotic that inhibits mycolic acid synthesis, i.e. in order to determine whether it acts on Pks13 or on an enzyme involved in the activation of one of its substrates.

This can be carried out by analyzing the fatty acids present in the C. glutamicum bacteria cultured in the presence of the xenobiotic (antibiotic candidate), for example by gas chromatography followed by mass spectrometry (GC-MS).

For this, methylated esters of fatty acids can be obtained by saponification of the cells, followed by methylation with diazomethane, as is described by Laval et al. (Annal. Chem., 2001, Vol. 73, p. 4537-4544). They are subsequently fractionated on a Florisil column irrigated with petroleum ether containing 0, 1, 2, 3 and 100% of diethyl ether. The methylated esters of polar fatty acids are contained in the last fraction eluted. Alternatively, it is possible to obtain trimethylsilylated derivatives by the method described by Constant et al. (J. Biol. Chem. 2002, Vol. 277, p., 38148-38158).

The analyses by gas chromatography and by gas chromatography followed by mass spectrometry can be carried out as described by Portevin et al. (PNAS 2004, above).

These analyses of the fatty acid content of the bacteria cultured in the presence and in the absence of the xenobiotic that inhibits mycolic acid synthesis make it possible to determine whether the xenobiotic acts on Pks13 or FadD32, or on the acyl carboxylase containing AccD4. The inhibition of the condensation by Pks13 or of the formation of acyl-AMP by FadD32 results in the accumulation of the intermediates resulting from the carboxylation by acyl-CoA carboxylase, such as tetradecylmalonic acid. The absence of accumulation of this compound indicates that the xenobiotic acts on the carboxylase containing. AccD4. In order to determine whether the xenobiotic acts on FadD32, a test can be carried out by purifying the FadD32 protein and measuring the formation of acyl-AMP in vitro, as described by Trivedi et al. (Nature 2004, Vol. 428, p. 441-445), in the presence or absence of the xenobiotic. The observation of an absence of acyl-AMP formation in the presence of the xenobiotic indicates that it acts on FadD32. The opposite result indicates that the xenobiotic acts on Pks13.

A bacterium in which the Pks13 gene has been mutated can serve as a control to verify the accumulation of these two substrates. For this, it is preferable to inactivate the Pks13 gene by means of a point mutation or a deletion, rather than by introducing a foreign sequence into the pks13 gene, as described above. This is because the introduction of the km cassette into the pks13 gene is capable of inducing a deficiency in expression of the accD4 gene in the mutant described above. Comparison of the spectra obtained with (i) C. glutamicum bacteria cultured in the absence of the xenobiotic, (ii) these same bacteria, cultured in the presence of the xenobiotic, (iii) C. glutamicum bacteria comprising a nonsense mutation in the pks13 gene, and, where appropriate, (iv) C. glutamicum bacteria in which the accD4 gene or the FadD32 gene has been mutated, makes it possible to determine whether the inhibition of mycolic acid synthesis by the xenobiotic is related to its action on Pks13, or on an enzyme located upstream in the biosynthesis of mycolic acids.

What we claim is:

1. A purified protein, comprising
a) at least 40% identity, over its entire sequence, with the Pks13 protein of M. tuberculosis (SEQ ID NO: 1); and
b) an acyltransferase domain (pfam00698), a keto acyl synthase domain (pfam02801 or pfam00109), at least one acyl carrier protein domain (COG0331 or COG0304), and a thioesterase domain (COG3319 or pfam00975); wherein
c) the purified protein catalyzes a Claisen condensation or malonic condensation between an acyl-CoA or acyl-AMP molecule and an acylmalonyl-CoA molecule.

2. The purified protein of claim 1, wherein the purified protein catalyzes a Claisen condensation or malonic condensation between:
a) an acyl-CoA molecule of formula I, or an acyl-AMP molecule of formula Ia: 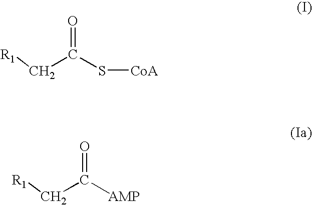
wherein R₁ is a chain comprising from 6 to 68 carbon atoms, which may comprise one or more C═C double bonds, one or more cis, trans, or cis and trans-cyclopropane rings, one or
more groups 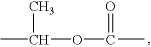
or a combination thereof, and which may carry one or more side groups selected from the group consisting of —CH₃, ═O and —O—CH₃;
and
b) an acylmalonyl-CoA molecule of formula II: 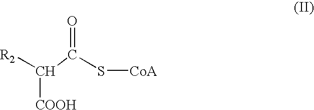
wherein R₂ is a linear alkane comprising from 10 to 24 carbon atoms;
so as to form a β-keto acyl intermediate of formula III, or a β-keto ester of formula IIIa: 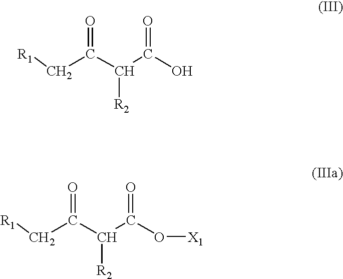
wherein R₁ and R₂ are as defined above, and X₁ is an acceptor molecule.

3. The purified protein of claim 1 comprising at least 70% identity with the sequence SEQ ID No.: 1 from Mycobacterium tuberculosis.

4. The protein of claim 2, further comprising at least 70% sequence identity with the sequence SEQ ID No.: 2 from Corynebacterium glutamicum.

5. An expression vector, comprising a polynucleotide sequence encoding the protein of claim 1.

6. A host cell transformed with the expression vector of claim 5.

7. The host cell of claim 6, wherein the host cell is a prokaryotic cell.

8. A method for obtaining a protein, wherein the protein comprises
a) at least 40% identity, over its entire sequence, with the Pks 13 protein of M. tuberculosis (SEQ ID NO: 1); and
b) an acyltransferase domain (pfam00698), a keto acyl synthase domain (pfam02801 or pfam00109), at least one acyl carrier protein domain (COG0331 or COG0304), and a thioesterase domain (COG3319 or pfam00975); wherein
c) the purified protein catalyzes a Claisen condensation or malonic condensation between an acyl-CoA or acyl-AMP molecule and an acylmalonyl-CoA molecule, comprising
culturing the host cell of claim 6; and
purifying the protein from the culture.

9. A method for inhibiting the biosynthesis of a mycolata envelope in a bacterium, comprising inhibiting, in the bacterium, the expression or the activity of the protein of claim 1, thereby inhibiting the mycolata envelope biosynthesis.

10. The purified protein of claim 1, wherein the purified protein catalyzes a Claisen condensation between the acyl-CoA molecule and the acylmalonyl-CoA molecule.

11. The purified protein of claim 1, wherein the purified protein catalyzes a Claisen condensation between the acyl-AMP molecule and the acylmalonyl-CoA molecule.

12. The purified protein of claim 1, wherein the purified protein catalyzes a malonic condensation between the acyl-CoA molecule and the acylmalonyl-CoA molecule.

13. The purified protein of claim 1, wherein the purified protein catalyzes a malonic condensation between the acyl-AMP molecule and the acylmalonyl-CoA molecule.

14. The purified protein of claim 2, wherein the purified protein catalyzes a Claisen condensation between the acyl-CoA molecule of formula I and the acylmalonyl-CoA molecule of formula II.

15. The purified protein of claim 2, wherein the purified protein catalyzes a Claisen condensation between the acyl-AMP molecule of formula Ia and the acylmalonyl-CoA molecule of formula II.

16. The purified protein of claim 2, wherein the purified protein catalyzes a malonic condensation between the acyl-CoA molecule of formula I and the acylmalonyl-CoA molecule of formula II.

17. The purified protein of claim 2, wherein the purified protein catalyzes a malonic condensation between the acyl-AMP molecule of formula Ia and the acylmalonyl-CoA molecule of formula II.

18. An expression vector comprising a polynucleotide sequence encoding the protein of claim 2.


SEQUENCE LISTING (31 sequences)

SEQ ID NO: 1 - protein, 1733 aa, Mycobacterium tuberculosis
MADVAESQENAPAERAELTVPEMRQWLRNWVGKAVGKAPDSIDESVPMVELGLSSRDAVAMAADIEDLTGVTLSVAVAFAHPTIESLATRIIEGEPETDLAGDDAEDWSRTGPAERVDIAIVGLSTRFPGEMNTPEQTWQALLEGRDGITDLPDGRWSEFLEEPRLAARVAGARTRGGYLKDIKGFDSEFFAVAKTEADNIDPQQRMALELTWEALEHARIPASSLRGQAVGVYIGSSTNDYSFLAVSDPTVAHPYAITGTSSSIIANRVSYFYDFHGPSVTIDTACSSSLVAIHQGVQALRNGEADVVVAGGVNALITPMVTLGFDEIGAVLAPDGRIKSFSADADGYTRSEGGGMLVLKRVDDARRDGDAILAVIAGSAVNHDGRSNGLIAPNQDAQADVLRRAYKDAGIDPRTVDYIEAHGTGTILGDPIEAEALGRVVGRGRPADRPALLGAVKTNVGHLESAAGAASMAKVVLALQHDKLPPSINFAGPSPYIDFDAMRLKMITTPTDWPRYGGYALAGVSSFGFGGANAHVVVREVLPRDVVEKEPEPEPEPKAAAEPAEAPTLAGHALRFDEFGNIITDSAVAEEPEPELPGVTEEALRLKEAALEELAAQEVTAPLVPLAVSAFLTSRKKAAAAELADWMQSPEGQASSLESIGRSLSRRNHGRSRAVVLAHDHDEAIKGLRAVAAGKQAPNVFSVDGPVTTGPVWVLAGFGAQHRKMGKSLYLRNEVFAAWIEKVDALVQDELGYSVLELILDDAQDYGIETTQVTIFAIQIALGELLRHHGAKPAAVIGQSLGEAASAYFAGGLSLRDATRAICSRSHLMGEGEAMLFGEYIRLMALVEYSADEIREVFSDFPDLEVCVYAAPTQTVIGGPPEQVDAILARAEAEGKFARKFATKGASHTSQMDPLLGELTAELQGIKPTSPTCGIFSTVHEGRYIKPGGEPIHDVEYWKKGLRHSVYFTHGIRNAVDSGHTTFLELAPNPVALMQVALTTADAGLHDAQLIPTLARKQDEVSSMVSTMAQLYVYGHDLDIRTLFSRASGPQDYANIPPTRFKRKEHWLPAHFSGDGSTYMPGTHVALPDGRHVWEYAPRDGNVDLAALVRAAAAHVLPDAQLTAAEQRAVPGDGARLVTTMTRHPGGASVQVHARIDESFTLVYDALVSRAGSESVLPTAVGAATAIAVADGAPVAPETPAEDADAETLSDSLTTRYMPSGMTRWSPDSGETIAERLGLIVGSAMGYEPEDLPWEVPLIELGLDSLMAVRIKNRVEYDFDLPPIQLTAVRDANLYNVEKLIEYAVEHRDEVQQLHEHQKTQTAEEIARAQAELLHGKVGKTEPVDSEAGVALPSPQNGEQPNPTGPALNVDVPPRDAAERVTFATWAIVTGKSPGGIFNELPRLDDEAAAKIAQRLSERAEGPITAEDVLTSSNIEALADKVRTYLEAGQIDGFVRTLRARPEAGGKVPVFVFHPAGGSTVVYEPLLGRLPADTPMYGFERVEGSIEERAQQYVPKLIEMQGDGPYVLVGWSLGGVLAYACAIGLRRLGKDVRFVGLIDAVRAGEEIPQTKEEIRKRWDRYAAFAEKTFNVTIPAIPYEQLEELDDEGQVRFVLDAVSQSGVQIPAGIIEHQRTSYLDNRAIDTAQIQPYDGHVTLYMADRYHDDAIMFEPRYAVRQPDGGWGEYVSDLEVVPIGGEHIQAIDEPIIAKVGEHMSRALGQIEADRTSEVGKQ

SEQ ID NO: 2 - protein, 1610 aa, Corynebacterium glutamicum
MEQSQSSDQKMTVEQVRTWLRDWVVRTTGIPVEEVTDDKAMETFGLSSRDVVVLSGELENLLDTSLDATIAYEYPTIRSLAQRLVEGEPRRAHTQRELNFSAVSDSPGSHDIAVVGMAARYPGAESLEDMWKLLVEGRDGISDLPIGRWSEYAGDEVMSRKMEEFSTIGGYLSDISSFDAEFFGLSPLEAANMDPQQRILLELTWEALEYARIAPNTLRGEAVGVFIGSSNNDYGMMIAADPAEAHPYALTGTSSAIVANRINYAFDFRGPSVNVDTACSSSLVAVHQAVRALRNGEADHAIAGGVNILASPFVTTAFAELGVISPTGKIHAFSDDADGFVRSDGAGVVVLKRVDDAIRDGDKIIGVIKGSAVNSDGHSNGLTAPNPDAQVDVLQRAYVDAQVDPTTVDYVEAHGTGTILGDPIEATALGAVLGYGRDASTPTLLGSAKSNFGHTESAAGIAGVIKVLLALQNKTLPPTVNFAGPNRYIDFDAERLEVVEDPREWPEYNGHAVAGVSAFGFGGTNAHVVISEYNAEDYETRAPKEALLPDQQVALPVSGHLPSRRRQAAADLADFLEGRKDCDLTPVARALAGRNHGRSRAVVLASTIEEAVKRLRQVAEGKVSVGISAADSPAANGPVFVYSGFGSQHRLMIKELCSISPQFRERIEELDEMVKFESGWSIMKLVLDDEQTYDTETAQVVITAIQIALTDLLASFGVKPAAVMGMSMGEIAAAYAAGGLSDRDTMLIASHRSRLMGEGEKSLAEDQLGAMAVVEFAAADLDKFIEENPEYKGIEPAVYAGPGMTTVGGPRDAVVQFVEKLESEDKFARLLNVKGAGHTSAVEPLLGELAGEIAGIEPLPLQIPLFSSVDQGVTYPVGAVVHDADYMLRCTRQSVYFQDSTEAAFAAGHNTLVEISPNPVALMGMMNTAFTVGKPDAQLLFSLKRKVPEAESLRDLLAKLYVNGANVDFSALYGEGETIDPPHITWKHQRFWTSARPSSGASLDLPGFRVNLPNNTVAFSTAAELAPSAVAIMEAAAMAVTPGSSVDAVDERDMLPPSGEITTIVTRSLGGLSLSVYKIEGTTSTLVAEGFAANPGFAAASSFDGPGYDGFNTDYSDQPDPRSDLPLDIEAVRWDPATETVEERMRAIVSEAMGYDVDDLPRELPLIDLGLDSLMGMRIKNRIENDFQIPPLQVQALRDASVADVVIMVENMVAGRSSETLVDATPQVPAEAAGEAQAAESSASGEDVQGVGVAPRDASERMVFGTWAGLTGAAAAGVTSKLPQIDVDTATAIAERLTERSGIEISTEQVLAAETLEPLSDLVREGLETEVQGNIRVLRGRAEGSTKPAVFMFHPAGGSSVVYQPLMRRLPEDVPVYGVERLEGDLADRAAAYVDDIKKYSDGFPVVLGGWSFGGAVAFEVAHQLVGSDVEVATVALLDTVQPSNPAPDTAEETRARWTRYADFAKKTYGLDFEVPFEILDTIGEDGMLSMMTDFLANTDASEHGLSAGVLEHQRASFVDNRILAKLNFADWANVEAPVILFRAERMHDGAIELEPNYAKIDQDGGWSGIVNDLEIVQLNGDHLAVVDEPEIGTVGAHLSRRIDEISRKN

SEQ ID NO: 3 - DNA, 21 bp, Artificial sequence
  PCR primer  pks13a
gctggarctvacvtgggargc

SEQ ID NO: 4 - DNA, 24 bp, Artificial sequence
  PCR primer  pks13b
gtgsgcgttggydccraavccgaa

SEQ ID NO: 5 - DNA, 28 bp, Artificial sequence
  PCR primer  13Rtb
gaggacatatggctgacgtagcggaatc

SEQ ID NO: 6 - DNA, 32 bp, Artificial sequence
  PCR primer  13Stb
cggtgaaagcttctgcttgcctacctcacttg

SEQ ID NO: 7 - DNA, 32 bp, Artificial sequence
  PCR primer  13Ttb
gctcggggatcctcactgcttgcctacctcac

SEQ ID NO: 8 - DNA, 33 bp, Artificial sequence
  PCR primer  13Ccg
aatatgactagtagccaatcgtcggatcagaag

SEQ ID NO: 9 - DNA, 35 bp, Artificial sequence
  PCR primer  13Dcg
agctctagatctctaattcttccgagaaatctcat

SEQ ID NO: 10 - DNA, 22 bp, Artificial sequence
  PCR primer  pkde15
gaaatctcgagccacggcgaaa

SEQ ID NO: 11 - DNA, 23 bp, Artificial sequence
  PCR primer  pkde12
acgattgccgcggttccatattg

SEQ ID NO: 12 - DNA, 24 bp, Artificial sequence
  PCR primer  pkde13
catcctgttccgcggaacgcatgc

SEQ ID NO: 13 - DNA, 23 bp, Artificial sequence
  PCR primer  pkde14
cagcatgatggagatctgagggc

SEQ ID NO: 14 - DNA, 21 bp, Artificial sequence
  PCR primer  fa2
tctgaccaccttccgtgaagc

SEQ ID NO: 15 - DNA, 18 bp, Artificial sequence
  PCR primer  ac2
gaacgagttcagagcttc

SEQ ID NO: 16 - DNA, 27 bp, Artificial sequence
  PCR primer  K10
tatttcgaatggttcgctgggtttatc

SEQ ID NO: 17 - DNA, 20 bp, Artificial sequence
  PCR primer  K7
taaaaagcttatcgataccg

SEQ ID NO: 18 - DNA, 18 bp, Artificial sequence
  PCR primer  pk1
gccgtgacggtatctcgg

SEQ ID NO: 19 - DNA, 20 bp, Artificial sequence
  PCR primer  pk2
ccagggcagttgcttcaatg

SEQ ID NO: 20 - DNA, 22 bp, Artificial sequence
  PCR primer  pk3
tccggaaagatctcacgccgcg

SEQ ID NO: 21 - DNA, 22 bp, Artificial sequence
  PCR primer  pk4
gcgtgcgcgcagatctgctagc

SEQ ID NO: 22 - DNA, 39 bp, Artificial sequence
  PCR primer  13F
gctctagagtttaaacgctggacctgtccaacgtcaagg

SEQ ID NO: 23 - DNA, 30 bp, Artificial sequence
  PCR primer  13G
ggactagtcgtcgaaaccgaccgtcaccag

SEQ ID NO: 24 - DNA, 28 bp, Artificial sequence
  PCR primer  13H
ggactagtcggcatcttcaacgagttgc

SEQ ID NO: 25 - DNA, 37 bp, Artificial sequence
  PCR primer  13I
cccaagcttgtttaaacttgtcgaagtggttcgacgg

SEQ ID NO: 26 - DNA, 20 bp, Artificial sequence
  PCR primer  13J
cttccacgacatggtctgat

SEQ ID NO: 27 - DNA, 20 bp, Artificial sequence
  PCR primer  13K
cacgatcgagtcgagctcga

SEQ ID NO: 28 - DNA, 19 bp, Artificial sequence
  PCR primer  H1
agcaccagcggttcgccgt

SEQ ID NO: 29 - DNA, 21 bp, Artificial sequence
  PCR primer  H2
tgcacgacttcgaggtgttcg

SEQ ID NO: 30 - DNA, 27 bp, Artificial sequence
  PCR primer  13R
atgagatctgatgaaaaccacagcgat

SEQ ID NO: 31 - DNA, 28 bp, Artificial sequence
  PCR primer  13P
ggactagtcttggcgacggccttctcac